(19) United States
(12) Patent Application Publication
(10) Pub. No.: US 2009/0215037 A1
(43) Pub. Date: Aug. 27, 2009

(54) Dynamically expressed genes with reduced redundancy

(75) Inventors: Xiao-Jun Ma, San Diego, CA (US); Xianqun Wang, San Marcos, CA (US); Mark G. Erlander, Encinitas, CA (US)

Correspondence Address: Patentique PLLC, P.O. Box 5803, Bellevue, WA, 98006, US

(73) Assignee: AviaraDx, Inc., Carlsbad, CA (US)

(21) Appl. No.: 11/816,623

(22) PCT Filed: Feb. 16, 2006
(86) PCT No.: PCT/US06/05552

§ 371 Date: Feb. 23, 2009

Related U.S. Application Data
(60) Provisional application No. 60/654,159, filed on Feb. 18, 2005.

(51) Int. Cl. C12Q 1/68; C40B 40/06

(52) U.S. Cl. 435/6; 506/16

(57) ABSTRACT

This invention relates to the identification and use of a subset of transcribed genes, wherein the expression of genes in the subset are able to classify cells among a plurality of classes. Methods for the identification or selection of such subsets are provided, along with computer implemented means for the application of the methods. The invention further provides physical embodiments based on the gene sequences of the subsets as well as methods for the use of the identified sets of gene sequences to classify a cell or tissue sample.

### RELATED APPLICATIONS

This application claims benefit of priority from Provisional U.S. Patent Application 60/654,159, filed Feb. 18, 2005, which is hereby incorporated in its entirety as if fully set forth.

### FIELD OF THE INVENTION

This invention relates to the identification and use of a subset of transcribed genes, wherein the expression of genes in the subset are able to classify cells among a plurality of classes. Methods for the identification or selection of such subsets are provided, along with computer implemented means for the application of the methods. The invention further provides physical embodiments based on the gene sequences of the subsets as well as methods for the use of the identified sets of gene sequences to classify a cell or tissue sample.

### BACKGROUND OF THE INVENTION

The concept of cellular phenotype includes, optionally in the aggregate, the characteristics of a cell. A cell's phenotype arises from the expression of gene sequences in its genome. The phenotype of a cell is responsible for the properties and/or capabilities of the cell. Examples of such properties include the cell or tissue type of a cell (e.g. muscle cell versus a secretory cell of the endocrine system versus an olfactory cell versus an immune system cell, while examples of such capabilities include the functionalities of what the cell does (e.g. a B cell versus a T cell in the immune system) as well as what the cell is capable of (e.g. an invasive as opposed to non-invasive tumor cell).

Each property or capability, as well as combination of property(ies) and capability(ies) may be considered a class into which a cell may be categorized or classified. The correct classification of a cell improves the ability to diagnose disease, determine or select treatment therefor, and/or predict the disease prognosis or outcome. Many classification methods are based in whole or in part on subjective visual evaluation of the appearance of a cell, in isolation or in situ, to evaluate morphology. Other methods include the use of chemical or immunohistochemical staining of cellular components. These methologies can result in unclear classifications, especially where similar morphology and/or staining are present for distinct disease conditions.

Given the availability of nucleic acid and protein array technologies, a cell's phenotype can potentially be determined based upon the expression of thousands of gene sequences. The particular combination of gene sequences that are expressed in a cell is believed to be highly relevant, as well as highly or nearly unique, to the classification of the cell based on one or more property and/or capability. Moreover, the determination of gene expression may be conducted in a quantitative manner to improve any classification methodology based thereon. As an initial approximation, it may be considered that the more expressed sequences evaluated, the greater the accuracy of predictions based on the expression of the evaluated sequences. This reflects the concept that greater numbers of sequences would provide greater amounts of information for distinguishing cells of one class from another. However, the evaluation of the expression of tens of thousands of gene sequences can be difficult and cumbersome.

Citation of documents herein is not intended as an admission that any is pertinent prior art. All statements as to the date or representation as to the contents of documents is based on the information available to the applicant and does not constitute any admission as to the correctness of the dates or contents of the documents.

### SUMMARY OF THE INVENTION

This invention relates to the identification and use of a subset of transcribed genes, wherein the expression of genes in the subset are able to classify cells among a plurality of classes. Methods for the identification or selection of such subsets, such as by removal or exclusion of gene identities from a larger set of expressed sequences, are provided, along with computer implemented means for the application of the methods. The invention further provides physical embodiments based on the gene sequences of the subsets as well as methods for the use of the identified sets of gene sequences to classify a cell or tissue sample as being of a particular class based on tissue source and/or cell or tumor type. They may also be used to assist in the determination of treatment and/or the prognosis of the subject from whom the sample was obtained.

In a first aspect, the invention provides a method of obtaining a set of gene sequences for use in classifying cells or cell containing tissues based upon the expression of said gene sequences. The method may be viewed as removing or excluding expressed gene sequences from a larger set, or as selecting gene sequences that are dynamically expressed across different cell phenotypes. The method has led to the discovery that a reduced number, or subset, of gene sequences provide the capability of defining the properties and/or characteristics (or phenotype) of a cell in which they are expressed. Stated differently, the reduced number, or subsets, of gene sequences disclosed herein are capable of defining, or classifying, the characteristics (or phenotype) defined by a larger set of genes, such as all the expressed gene sequences (or transcriptome) of a cell. Thus the invention is based in part on the surprising discovery that the information regarding the expression levels of a large number of sequences in the transcriptome of a cell are redundant in their ability to provide information on the cellular properties and/or characteristics (or phenotype) of a cell in which they are expressed.

The present invention may thus be viewed as providing methods of conducting feature selection by reducing the amount of redundant gene expression information to be evaluated. Feature selection may be considered the selection of relevant genes for use in classification. This is desirable at least because it provides the ability to analyze a subset of gene expression instead of a larger set of expressed genes, or the entire set of expressed sequences (or “transcriptome”). Feature selection also permits a small set of relevant gene sequences to be the focus for developing a diagnostic tool based on classification. These methods of the invention do not “search” through the space of possible data on the expression of individual genes or combinations thereof for those that may be useful in classification. That type of methodology is based on the points in the space reflecting possible candidate expression patterns or profiles for use in classification with the assumption that one or more of the candidate expression patterns will satisfy the goal of being able to classify among a given group of classes or categories. The methods of the invention are instead directed to two goals: remove redundant gene expression information and select for genes that are dynamically expressed across a variety of cell phenotypes. The methods also provide benefits by reducing “noise” and/or over representation by genes expressed in correlation with the same property and/or characteristic (or phenotype).

Thus, the methods of the invention to reduce the number of gene sequences required to classify a cell also do not include prior assignment of the expression data for each gene sequence to a property and/or characteristic (or phenotype) of a cell. Accordingly, no bias with regard to gene sequences expressed in correlation with a class used in the methods was present. Instead, gene sequences which were expressed in redundant fashion with other gene sequences were identified and excluded, based in part upon their range of variability, to reduce the number of gene sequences. The methods led to the unexpected discovery that expression of the resultant subset of gene sequences can be used for classification with accuracy equal to or greater than that seen with a larger, more redundant and less variable set of gene sequences. Additionally, the subsets were able to classify additional classes based on a property and/or characteristic beyond that of the cells used to identify the subset.

Thus in a second aspect of the invention, a subset of gene sequences, expressed in a cell, is provided wherein the subset has reduced redundancy and increased variability while retaining the ability to classify a cell based on a property and/or characteristic, including a property and/or characteristic beyond those of the cells used to identify the subset of gene sequences. The subset of gene sequences may also be considered a gene set, comprising a fraction, or subset, of all transcripts expressed by a cell, that provides information useful for classification.

In a third aspect, a subset of gene sequences expressed in a cell, as provided by the invention, may be used in a method of classifying a cell containing test sample from a subject among classes in a training set of samples, of known classes, by comparing expression of the subset of gene sequences in the cell containing test sample to expression of said gene sequences in the training set of samples. The comparison can then be used to classifying the test sample as being of one of the known classes, even where the known classes includes one or more that were not used in the methods to arrive at the subset. This latter ability of a subset of the invention indicates that expression data of the gene sequences in the subset is informative, via variability in expression, for classification beyond the classes used to identify the subset. Accordingly, the subset may be used in a “generic” manner to classify between some or all of the classes used to identify the subset as well as classify between additional classes beyond the classes used to identify the subset. Classification between classes used to identify the subset and additional classes beyond them can also be conducted.

The analysis of gene expression from a subset, or reduced number, of gene sequences may be considered a quantitative phenotyping tool such that the expression data of the subset is an expression profile, expression signature or expression phenotype useful for classifying a cell, including a tumor cell or tissue, or other disease cell or tissue, into different categories. Non-limiting examples of such classes (or categories) include different cell or tissue type origins, different subtypes of tumor with the same origin, cancer versus non-cancer, and/or clinically relevant classes. Non-limiting examples of clinically relevant classes include responsiveness (or lack thereof) to various treatments (such as drugs or radiation), clinical course of a disease, treatment outcomes, and/or survival or prognosis of a subject.

In some embodiments, use of a subset of gene sequences for classification is via a classification algorithm used to apply supervised learning to evaluate the gene expression data of sequences in the subset as found in a training set of cell containing samples. Any appropriate supervised learning algorithm may be used in the practice of the invention. Non-limiting examples include K nearest neighbor (KNN); supported vector machine (SVM); canonical discriminant analysis (CDA); neural network (NN) or neural net (NN), including an artificial neural network (ANN); soft independent modeling of class analogy (SIMCA); linear discriminant analysis (LDA), including a maximum likelihood classifier like MLHD (see Ooi et al. as cited herein); decision trees, and any variant of the above. The classification algorithm serves as a means to apply the gene expression data from the training set to classifying a given sample, such as an unknown or test sample from a subject. Alternatively, a non-supervised algorithm, such as one based on clustering, may be used.

The classification algorithm may also be used to evaluate the performance of a subset in any suitable mode. Non-limiting examples include leave-one-out cross validation (LOOCV) among the training samples or the use of independent training and test samples (of known classes) to determine performance. In LOOCV, one sample is excluded from the training set and a new classifier is constructed based on the remaining samples. The new classifier is thus independent of the excluded sample, which is then used as the test sample for evaluating performance of the new classifier. This process is repeated for all of the training samples to determine performance of gene expression data based on the subset. In independent training and testing, the samples, which may be of a single initial group, are divided into a training group and a test group. Alternatively, one group of samples may be used as a training group and an independent second group of known samples may be used as a test group. The training group is used to build a classifier, and then performance is evaluated on the test group.

The invention is not limited to a single subset of gene sequences because the recognition of redundancy in the information provided by the expression levels of gene sequences expressed in a cell can be used to substitute one gene sequence of a subset with another gene sequence which provides the same information for classification. Thus the invention provides for multiple subsets of gene sequences, which may be embodied in a number of different formats for use in classification methods.

The invention also provides computer related means and systems, such as the embodiment shown in FIG. 4, for performing the methods provided herein. In some embodiments, an apparatus for obtaining a subset of gene sequences of the invention is provided. Such an apparatus may comprise a query storage configured to store gene expression data from a large set of gene sequences for reduction, as described herein, received from a query input; and a module for accessing and using data from the storage in a gene reduction algorithm, or method, as described herein. The apparatus may further comprise a string storage for the results of the gene reduction algorithm, or method, optionally with a module for accessing and using data from the string storage in a classification algorithm, or method, as described herein.

For use in classification, the invention provides computer related means and systems, like the embodiment shown in FIG. 4, for performing the subset based classification methods provided herein. In some embodiments, an apparatus for classification using subset of gene sequences of the invention is provided. Such an apparatus may comprise a query storage configured to store gene expression data from a subset of gene sequences, as described herein, received from a query input; and a module for accessing and using data from the storage in a classification algorithm, or method, as described herein. The apparatus may further comprise a string storage for the results of the classification algorithm, or method, optionally with a module for accessing and using data from the string storage in a classification algorithm, or method, as described herein.

The invention also provides one or more processor readable storage devices containing one or more processor usable instructions, when executed by one or more processors, to perform the method comprising allowing input of a gene expression data set of a large number of gene sequences obtained from a plurality of cell containing samples of known classes; and applying said expression data set to a gene reduction algorithm, or method, to result in a subset of gene sequences for use in classification. Additionally, the invention provides one or more processor readable storage devices containing one or more processor usable instructions, when executed by one or more processors, to perform a classification method comprising allowing input of a gene expression data set of a subset of gene sequences of the invention obtained from a plurality of cell containing samples of known classes; and applying said expression data set to a classification algorithm, or method, for use in classification of another sample, such as a cell containing sample from a subject, based on expression of all or part of the sequences in the subset. In some embodiments, the algorithms, or methods, are written in the R language.

The invention further provides a computer readable medium having stored thereon instructions configured to cause reduction of a set of gene sequences, based on the expression thereof in a plurality of known samples, to result in a reduced subset of gene sequences, the expression of which are useful in classifying a cell containing test sample among known classes, the instructions comprising program code for performing the methods described herein. In some embodiments, the instructions comprise program code for receiving a gene expression data set obtained from a plurality of samples of known classes; and program code for applying said expression data set to a gene reduction algorithm, to produce a reduced subset of gene sequences.

Similarly, the invention provides a computer readable medium having stored thereon instructions configured to cause classification of a cell containing sample from a subject, based on the expression, of a subset of gene sequences of the invention, in a plurality of known samples, to result in a classification model which is useful in classifying a cell containing test sample among known classes, the instructions comprising program code for performing the methods described herein. In some embodiments, the instructions comprise program code for receiving the gene expression data of a subset of the invention obtained from a plurality of samples of known classes; and program code for applying said expression data to a classification algorithm, for classifying another sample, such as a cell containing sample from a subject, based on expression of all or part of the sequences in the subset.

The steps of a method, process, or algorithm described in connection with the embodiments disclosed herein may be embodied directly in hardware, in a software module executed by a processor, or in a combination of the two. The various steps or acts in a method or process may be performed in the order shown, or may be performed in another order. Additionally, one or more process or method steps may be omitted or one or more process or method steps may be added to the methods and processes. An additional step, block, or action may be added in the beginning, end, or intervening existing elements of the methods and processes.

The details of one or more embodiments of the invention are set forth in the accompanying drawing and the description below. Other features, objects, and advantages of the invention will be apparent from the drawing and detailed description, and from the claims.

### DEFINITIONS

As used herein, a “gene” is a polynucleotide that encodes a discrete product, whether RNA or proteinaceous in nature. It is appreciated that more than one polynucleotide may be capable of encoding a discrete product. The term includes alleles and polymorphisms of a gene that encodes the same product, or a functionally associated (including gain, loss, or modulation of function) analog thereof, based upon chromosomal location and ability to recombine during normal mitosis.

A “sequence” or “gene sequence” as used herein is a nucleic acid molecule or polynucleotide composed of a discrete order of nucleotide bases. The term includes the ordering of bases that encodes a discrete product (i.e. “coding region”), whether RNA or proteinaceous in nature. It is appreciated that more than one polynucleotide may be capable of encoding a discrete product. It is also appreciated that alleles and polymorphisms of the human gene sequences may exist and may be used in the practice of the invention to identify the expression level(s) of the gene sequences or an allele or polymorphism thereof. Identification of an allele or polymorphism depends in part upon chromosomal location and ability to recombine during mitosis.

A “polynucleotide” is a polymeric form of nucleotides of any length, either ribonucleotides or deoxyribonucleotides. This term refers only to the primary structure of the molecule. Thus, this term includes double- and single-stranded DNA and RNA. It also includes known types of modifications including labels known in the art, methylation, “caps”, substitution of one or more of the naturally occurring nucleotides with an analog, and internucleotide modifications such as uncharged linkages (e.g., phosphorothioates, phosphorodithioates, etc.), as well as unmodified forms of the polynucleotide.

The term “amplify” is used in the broad sense to mean creating an amplification product can be made enzymatically with DNA or RNA polymerases. “Amplification,” as used herein, generally refers to the process of producing multiple copies of a desired sequence, particularly those of a sample. “Multiple copies” mean at least 2 copies. A “copy” does not necessarily mean perfect sequence complementarity or identity to the template sequence. Methods for amplifying mRNA are generally known in the art, and include reverse transcription PCR (RT-PCR) and quantitative PCR (or Q-PCR) or real time PCR. Alternatively, RNA may be directly labeled as the corresponding cDNA by methods known in the art.

By “corresponding”, it is meant that a nucleic acid molecule shares a substantial amount of sequence identity with another nucleic acid molecule. Substantial amount means at least 95%, usually at least 98% and more usually at least 99%, and sequence identity is determined using the BLAST algorithm, as described in Altschul et al. (1990), J. Mol. Biol. 215:403-410 (using the published default setting, i.e. parameters w=4, t=17).

A “microarray” is a linear or two-dimensional or three dimensional (and solid phase) array of preferably discrete regions, each having a defined area, formed on the surface of a solid support such as, but not limited to, glass, plastic, or synthetic membrane. The density of the discrete regions on a microarray is determined by the total numbers of immobilized polynucleotides to be detected on the surface of a single solid phase support, such as of at least about 50/cm², at least about 100/cm², at least about 500/cm², or below about 1,000/cm² as non-limiting examples. In some embodiments, the arrays contain less than about 500, about 1000, about 1500, about 2000, about 2500, or about 3000 immobilized polynucleotides in total. As used herein, a DNA microarray is an array of oligonucleotide or polynucleotide probes placed on a chip or other surfaces used to hybridize to amplified or cloned polynucleotides from a sample. Since the position of each particular group of probes in the array is known, the identities of a sample polynucleotides can be determined based on their binding to a particular position in the microarray. As an alternative to the use of a microarray, an array of any size may be used in the practice of the invention, including an arrangement of one or more position of a two-dimensional or three dimensional arrangement in a solid phase to detect expression of a single gene sequence. Bead arrays (such as those of Illunina Inc.) may also be used in embodiments of the invention. Software for analysis of microarray data is discussed by Dudoit et al. Biotechniques Suppl. 45-51 (March 2003).

Because the invention relies upon the identification of gene expression, some embodiments of the invention determine expression by hybridization of mRNA, or an amplified or cloned version thereof, of a sample cell to a polynucleotide that is unique to a particular gene sequence. Polynucleotides of this type contain at least about 16, at least about 18, at least about 20, at least about 22, at least about 24, at least about 26, at least about 28, at least about 30, or at least about 32 consecutive basepairs (as non-limiting examples) of a gene sequence that is not found in other gene sequences. The term “about” as used in the previous sentence refers to an increase or decrease of 1 from the stated numerical value. Other embodiments include polynucleotides of at least or about 50, at least or about 100, at least about or 150, at least or about 200, at least or about 250, at least or about 300, at least or about 350, at least or about 400, at least or about 450, or at least or about 500 consecutive bases of a sequence that is not found in other gene sequences. The term “about” as used in the preceding sentence refers to an increase or decrease of 10% from the stated numerical value. Longer polynucleotides may of course contain minor mismatches (e.g. via the presence of mutations) which do not affect hybridization to the nucleic acids of a sample. Such polynucleotides may also be referred to as polynucleotide probes that are capable of hybridizing to sequences of the genes, or unique portions thereof, described herein. Such polynucleotides may be labeled to assist in their detection. In some embodiments, the sequences are those of mRNA encoded by the genes, the corresponding cDNA to such mRNAs, and/or amplified versions of such sequences. In other embodiments of the invention, the polynucleotide probes are immobilized on an array, other solid support devices, or in individual spots that localize the probes.

In other embodiments of the invention, all or part of a gene sequence may be amplified and detected by methods such as the polymerase chain reaction (PCR) and variations thereof, such as, but not limited to, quantitative PCR (Q-PCR), reverse transcription PCR (RT-PCR), and real-time PCR (including as a means of measuring the initial amounts of mRNA copies for each sequence in a sample), optionally real-time RT-PCR or real-time Q-PCR. Such methods would utilize one or two primers that are complementary to portions of a gene sequence, where the primers are used to prime nucleic acid synthesis. The newly synthesized nucleic acids are optionally labeled and may be detected directly or by hybridization to a polynucleotide of the invention. The newly synthesized nucleic acids may be contacted with polynucleotides (containing sequences) of the invention under conditions which allow for their hybridization. Additional methods to detect the expression of expressed nucleic acids include RNAse protection assays, including liquid phase hybridizations, and in situ hybridization of cells.

As used herein, a “tumor sample” or “tumor containing sample” or “tumor cell containing sample” or variations thereof, refer to cell containing samples of tissue or fluid isolated from an individual suspected of being afflicted with, or at risk of developing, cancer. The samples may contain tumor cells which may be isolated by known methods or other appropriate methods as deemed desirable by the skilled practitioner. These include, but are not limited to, microdissection, laser capture microdissection (LCM), or laser microdissection (LMD) before use in the instant invention. Alternatively, undissected cells within a “section” of tissue may be used. Non-limiting examples of such samples include primary isolates (in contrast to cultured cells) and may be collected by any non-invasive or minimally invasive means, including, but not limited to, ductal lavage, fine needle aspiration, needle biopsy, the devices and methods described in U.S. Pat. No. 6,328,709, or any other suitable means recognized in the art. Alternatively, the sample may be collected by an invasive method, including, but not limited to, surgical biopsy.

The terms “label” or “labeled” refer to a composition capable of producing a detectable signal indicative of the presence of the labeled molecule. Suitable labels include radioisotopes, nucleotide chromophores, enzymes, substrates, fluorescent molecules, chemiluminescent moieties, magnetic particles, bioluminescent moieties, and the like. As such, a label is any composition detectable by spectroscopic, photochemical, biochemical, immunochemical, electrical, optical or chemical means.

The term “support” refers to conventional supports such as beads, particles, dipsticks, fibers, filters, membranes and silane or silicate supports such as glass slides.

“Expression” and “gene expression” include transcription and/or translation of nucleic acid material.

As used herein, the term “comprising” and its cognates are used in their inclusive sense; that is, equivalent to the term “including” and its corresponding cognates.

Conditions that “allow” an event to occur or conditions that are “suitable” for an event to occur, such as hybridization, strand extension, and the like, or “suitable” conditions are conditions that do not prevent such events from occurring. Thus, these conditions permit, enhance, facilitate, and/or are conducive to the event. Such conditions, known in the art and described herein, depend upon, for example, the nature of the nucleotide sequence, temperature, and buffer conditions. These conditions also depend on what event is desired, such as hybridization, cleavage, strand extension or transcription.

Sequence “mutation,” as used herein, refers to any sequence alteration in the sequence of a gene disclosed herein interest in comparison to a reference sequence. A sequence mutation includes single nucleotide changes, or alterations of more than one nucleotide in a sequence, due to mechanisms such as substitution, deletion or insertion. Single nucleotide polymorphism (SNP) is also a sequence mutation as used herein. Because the present invention is based on the relative level of gene expression, mutations in coding and non-coding regions of genes as disclosed herein may also be assayed for use in the practice of the invention.

“Detection” or “detecting” includes any means of detecting, including direct and indirect determination of the level of gene expression and changes therein.

Unless defined otherwise all technical and scientific terms used herein have the same meaning as commonly understood to one of ordinary skill in the art to which this invention belongs.

### BRIEF DESCRIPTION OF THE DRAWINGS

FIGS. 1A-1D show a “tree” that classifies a plurality of tumor types including those discussed herein. It was constructed mainly according to “Cancer, Principles and Practice of Oncology, (DeVito, Hellman and Rosenberg), 6^{th} edition”. Thus beginning with a “tumor of unknown origin” (or “TUO”), the first possibilities are that it is either of a germ cell or non-germ cell origin. If it is the former, then it may be of ovary or testes origin. Within those of testes origin, the tumor may be of seminoma origin or an “other” origin.

If the tumor is of a non-germ cell origin, then it is either of a epithelial or non-epithelial origin. If it is the former, then it is either squamous or non-squamous origin. Squamous origin tumors are of cervix, esophagus, larynx, lung, or skin in origin. Non-squamous origin tumors are of urinary bladder, breast, carcinoid-intestine, cholangiocarcinoma, digestive, kidney, liver, lung, prostate, reproductive system, skin-basal cell, or thyroid-follicular-papillary origin. Among those of digestive origin, the tumors are of small and large bowel, stomach-adenocarcinoma, bile duct, esophagus, gall bladder, and pancreas in origin. The esophagus origin tumors may be of either Barrett's esophagus or adenocarcinoma types. Of the reproductive system origin tumors, they may be of cervix adenocarcinoma type, endometrial tumor, or ovarian origin. Ovarian origin tumors are of the clear, serous, mucinous, and endometroid types.

If the tumor is of non-epithelial origin, then it is of adrenal gland, brain, GIST (gastrointestinal stromal tumor), lymphoma, meningioma, mesothelioma, sarcoma, skin melanoma, or thyroid-medullary origin. Of the lymphomas, they are B cell, Hodgkin's, or T cell type. Of the sarcomas, they are leimyosarcoma, osteosarcoma, soft-tissue sarcoma, soft tissue MFH (malignant fibrous histiocytoma), soft tissue sarcoma synovial, soft tissue Ewing's sarcoma, soft tissue fibrosarcoma, and soft tissue rhabdomyosarcoma types.

FIG. 2 shows the variance distribution comparison between expression levels of a set of 1935 gene sequences selected by a method of the invention (“Corrtrim”) and the expression levels of all gene sequences used in the gene expression data. While Corrtrim did not pick gene sequences expressed with high variance per se, in general, the variance is higher with Corrtrim, although most of the selected gene sequences have low variance (highest density with variance below 5).

FIG. 3 shows the results of a comparison between a gene set identification method of the invention (“Corrtrim”) as reflected in Table 1 herein and random selection of gene sequences based on the expression levels of 16,948 gene sequences. The results used both cross validation (CV) and prediction of a separate training set.

FIG. 4 shows the results of using “Corrtrim” with four successive sets (Rounds 1, 2, 3, and 4) of removing k gene sequences.

FIG. 5 is a functional block diagram of an example of a computer 200 which can be configured for use in the practice of the invention. The computer 200 can include a display 210, a processor 220, memory 224, a communication device 230, a network interface Card (NIC) 234, an I/O controller 240, a hard drive 262, one or more removable storage drives 264, which can include a floppy drive, and an optical storage 266, and one or more storage devices 268. The I/O controller 240 can be configured to interface with one or more I/O devices 250, which can include a keyboard 252 and some other input device 254. The NIC 234 can couple the computer 200 to a network, such as a local network of more than one computers or a larger network such as the internet as non-limiting examples.

### DETAILED DESCRIPTION OF MODES OF PRACTICING THE INVENTION

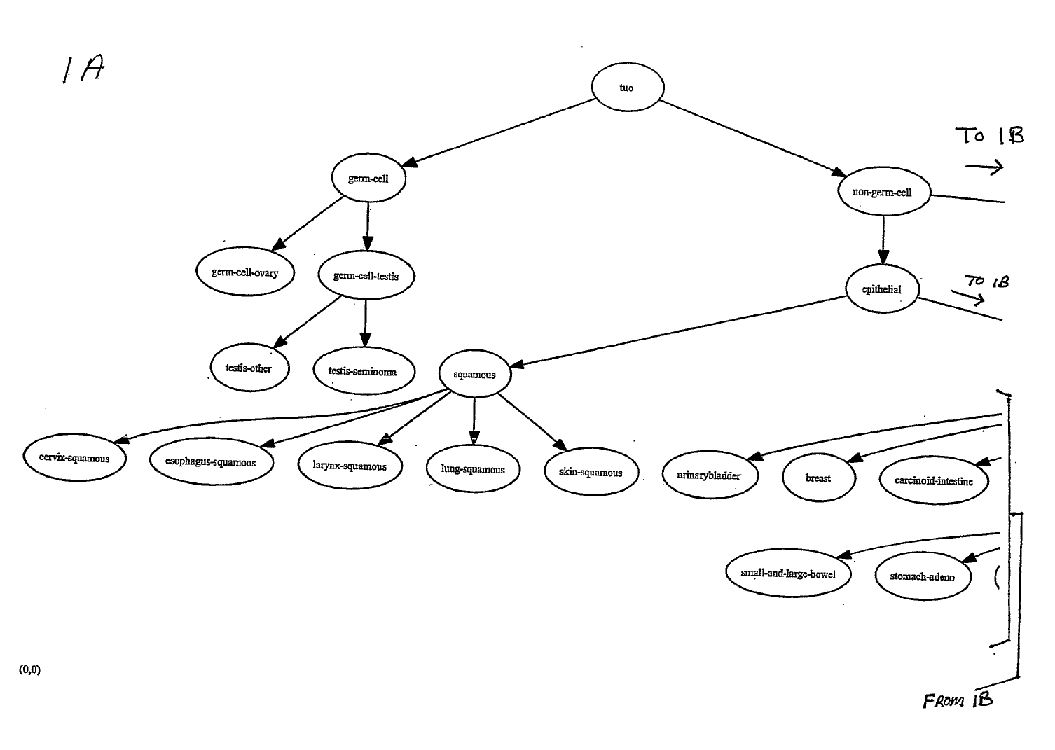

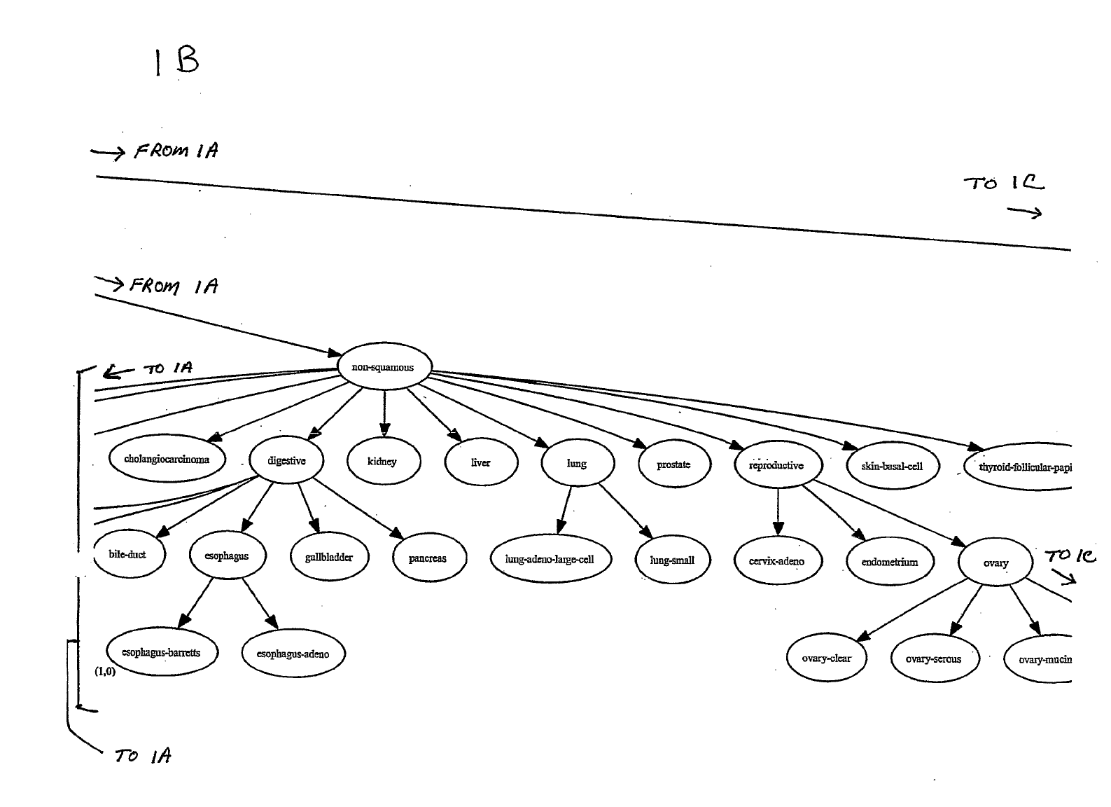

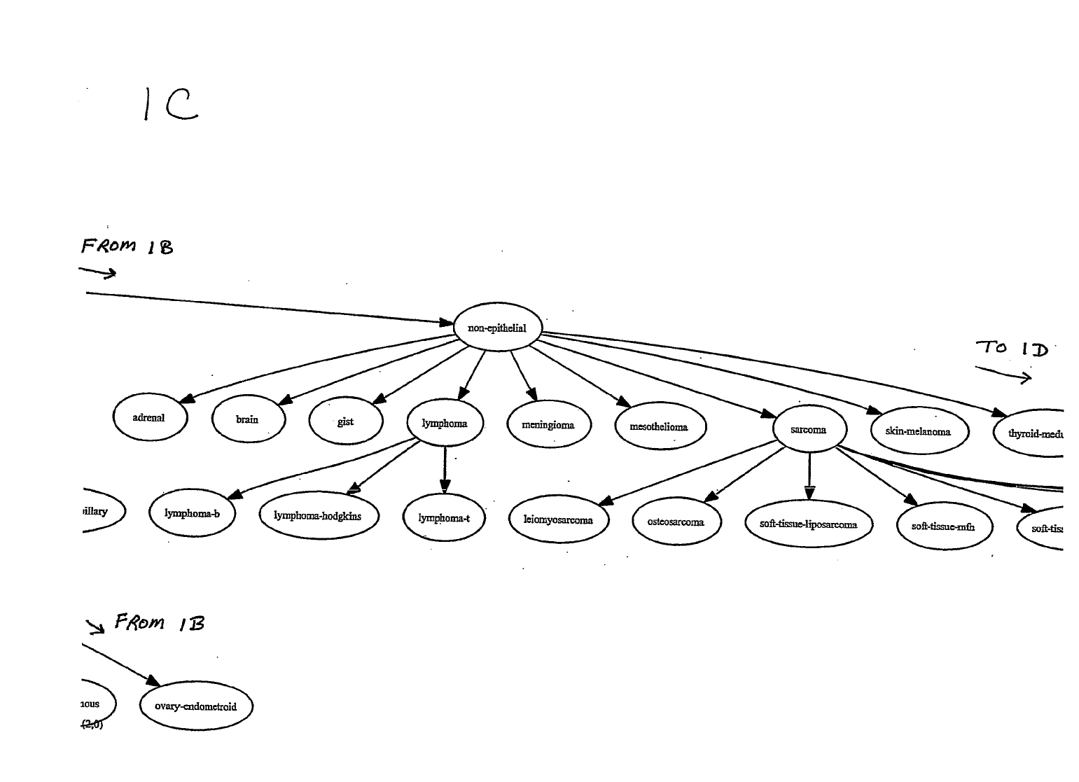

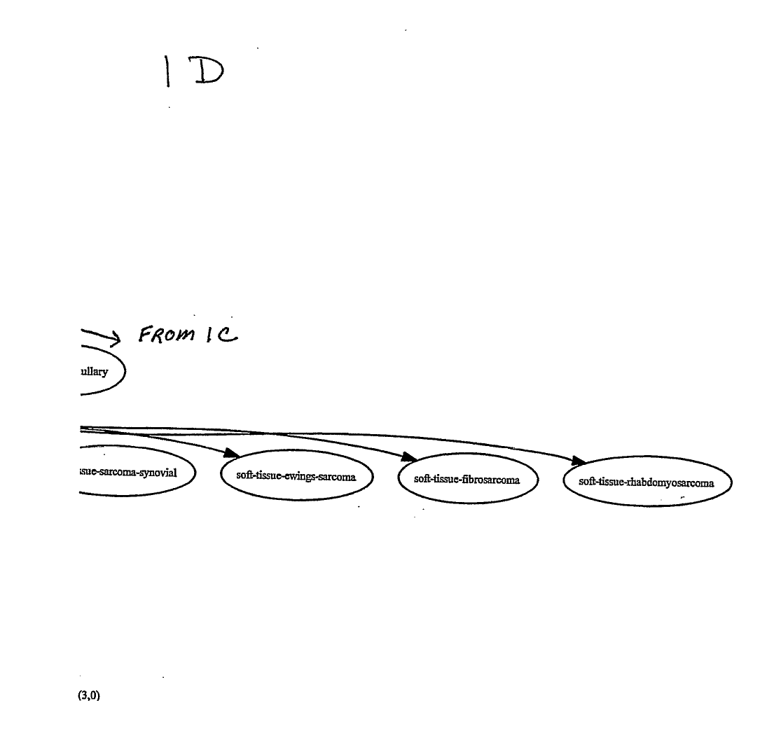

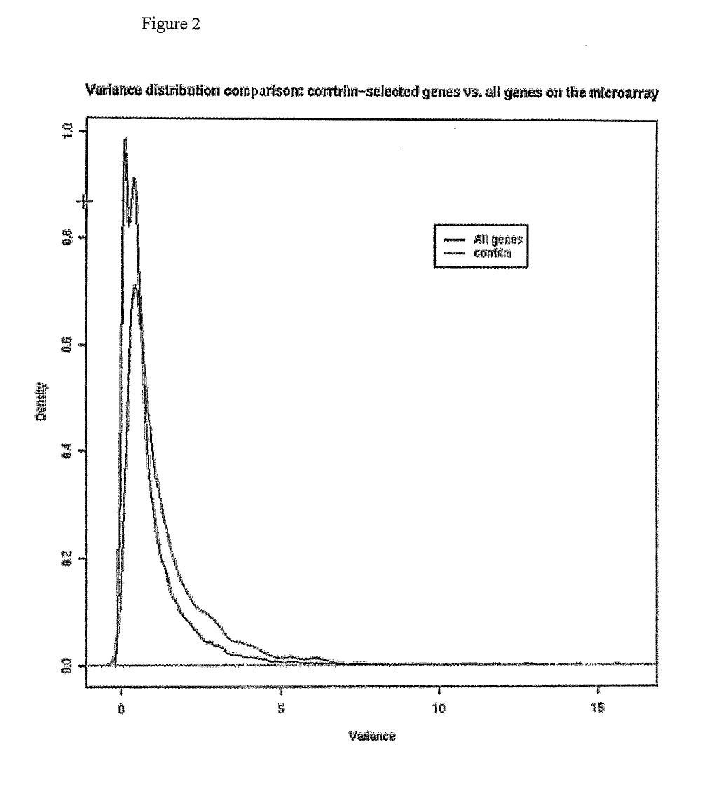

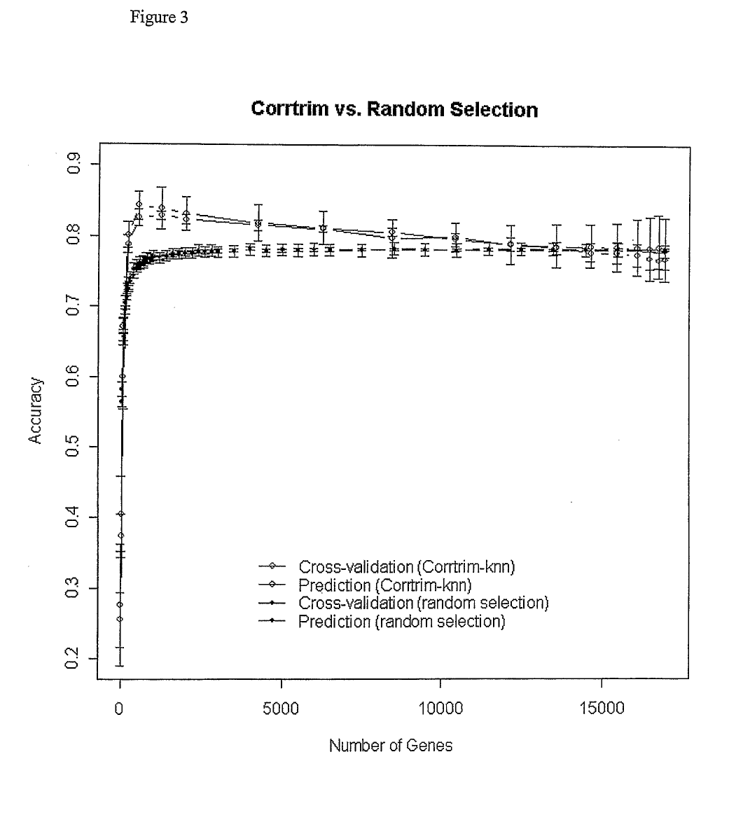

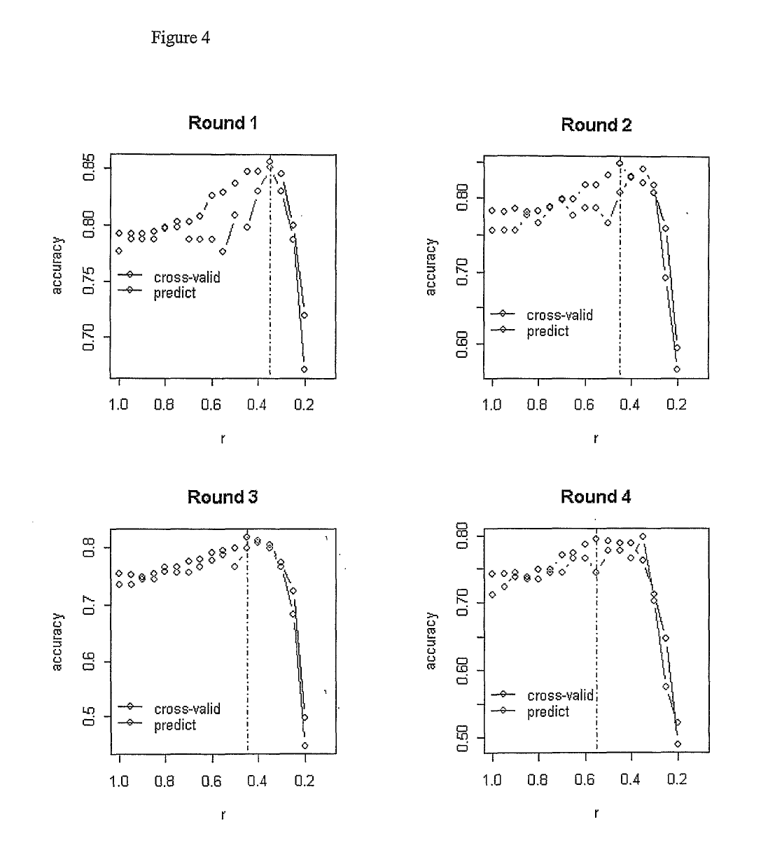

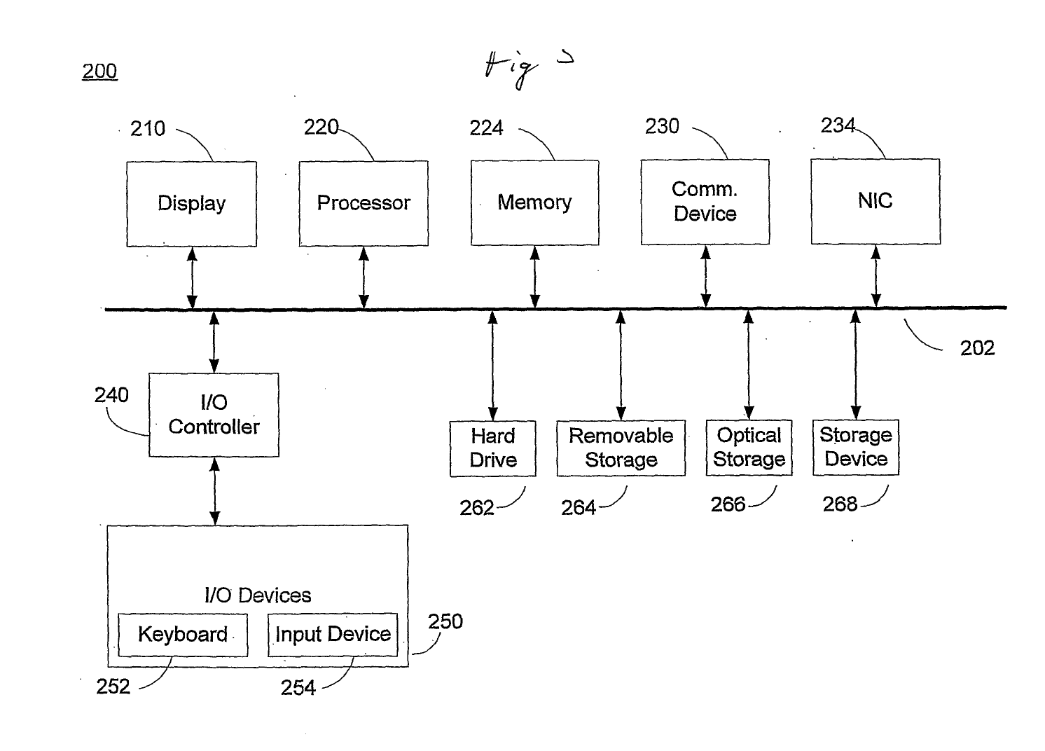

In one aspect, the invention provides a method of determining or identifying a subset of gene sequences, the expression of which are useful in classifying a cell or a cell containing test sample among known classes. The subset may be used in classifying cells or cell containing tissues based upon the expression some or all of the gene sequences in the subset. Such a method includes providing a gene expression data set of gene sequences expressed in a plurality of cell containing samples of a plurality of known classes. The plurality of known classes is preferably at least about 10, at least about 15, at least about 20, at least about 30, or at least about 40 or more different types, such as those reflected in the “tree” in FIG. 1. The actual number of sample types reflects the coverage which the plurality of samples provides. Thus any number of samples may be used so long as at least the genes expressed across the different sample types are sufficient to permit the identification of gene sequences that are dynamically expressed across the different sample types such that the expression level of the gene sequences can be used for classification at least among the sample types after selection according to the methods of the invention. In some embodiments, the number of sample types are sufficient to permit the identified gene sequences to be dynamically expressed across samples of cell phenotypes beyond those used in the plurality of known classes. One embodiment of the method is referred to as “Corrtrim” herein.

In some embodiments, the gene expression data set includes all sequences expressed in all, or most, of the cells in the plurality of samples. Such a data set would thus be of all or most of the “transcriptome” in the cells of the plurality of samples. In other embodiments, the gene expression data set would be of at least about 50% of the sequences expressed in the cells of each of the plurality of samples. The ability to derive a meaningful and useful subset from about 50% or more of the expressed sequences is provided by the discovery of the amount of redundancy in the information contained in gene expression data, where nearly two-thirds or more of the expression data is redundant in nature as discussed below. Thus the method may also be practiced with the use of an initial gene expression data set of at least about 60%, at least about 70%, at least about 80%, at least about 90%, or more of the genes expressed in the plurality of samples.

The method also comprises analyzing the gene expression data to determine the range of variability in expression of each gene sequence across the plurality of samples, and thus across the plurality sample types used. This facilitates subsequent exclusion, removal, or elimination of genes expressed with low variability as described below. One non-limiting means to determine the range of variability is by the calculation of variance in the expression of each gene sequence across the plurality of samples. It is pointed out, however, that the method of the invention is not directed to selection of gene sequences expressed with high variability per se. To the contrary, the method is based on the selection of sequences expressed with high variability relative to other gene sequences expressed in correlation as described below. Thus a gene expressed with relatively low variance in a comparison with all other genes may still be retained by the method if the gene is not correlated with any other gene or only correlated with other genes expressed with less variability. In practice, the gene sequences of a subset of the invention are expressed with relatively low variance when compared to the expression of other sequences. See FIG. 2.

The expression level of each gene sequence in the expression data set is also correlated, across the plurality of samples, with the expression level of each other gene in the data set to produce a correlation matrix of correlation coefficients. These correlation determinations may be performed directly, between expression of each pair of gene sequences, or indirectly, without direct comparison between the expression values of each pair of gene sequences. A variety of correlation methodologies may be used in the correlation of expression data of individual gene sequences within the data set. Non-limiting examples include parametric and non-parametric methods as well as methodologies based on mutual information and non-linear approaches. Non-limiting examples of parametric approaches include Pearson correlation (or Pearson r, also referred to as linear or product-moment correlation) and cosine correlation. Non-limiting examples of non-parametric methods include Spearman's R (or rank-order) correlation, Kendall's Tau correlation, and the Gamma statistic.

Each correlation methodology can be used to determine the level of correlation between the expressions of individual gene sequences in the data set. The level of correlation of all sequences with all other sequences is most readily considered as a matrix. Using Pearson's correlation as a non-limiting example, the correlation coefficient r in the method is used as the indicator of the level of correlation. When other correlation methods are used, the correlation coefficient analogous to r may be used, along with the recognition of equivalent levels of correlation corresponding to r being at or about 0.25 to being at or about 0.5.

The correlation coefficient may be selected as desired to reduce the number of correlated gene sequences to various numbers. In some embodiments of the invention using r, the selected coefficient value may be of about 0.25 or higher, about 0.3 or higher, about 0.35 or higher, about 0.4 or higher, about 0.45 or higher, or about 0.5 or higher. The selection of a coefficient value means that where expression between gene sequences in the data set is correlated at that value or higher, they are possibly not included in a subset of the invention. The gene sequences of a subset of the invention includes those which are expressed in correlation below the desired correlation coefficient value with another gene sequence and expressed with greater variability, or variance, than that other gene sequence. Thus in some embodiments, the method comprises excluding or removing one or more gene sequences that are i) expressed in correlation, above a desired correlation coefficient, with an other gene sequence in the data set, and ii) expressed with lower variance than said other gene sequence from inclusion in the subset. Alternatively, the method may comprise selecting those gene sequences that are i) expressed in correlation, below a desired correlation coefficient, with an other gene sequence in the data set, and ii) expressed with greater variability variance than said other gene sequence for inclusion in the subset. It is pointed out, however, that there can be situations of gene sequences that are not correlated with any other gene sequences, in which case they are not removed to result in their inclusion in the subset.

The removal or selection of gene sequences based on the above results in a subset of gene sequences expressed with greater variability, or higher variance, than other gene sequences expressed in correlation with said subset of gene sequences. It is this subset of gene sequences that can classify cells among a multitude of classes, including the plurality of classes from which the original gene expression data set was obtained. Alternatively, the subset can be used to classify among classes including one or more other classes not present in the original gene expression data set. Thus the gene sequences in the subset include those that are dynamically expressed across a plurality of different cell phenotypes such that the expression thereof can be used for classification among those phenotypes (or classes).

The ability to classify among additional classes not included as part of the original gene expression data set is provided by the expression of gene sequences in the subset as reflecting all, or the majority, of the properties and/or characteristics (or phenotype) of a cell. This follows because the gene sequences in the subset are correlated with the gene sequences excluded from the subset. Thus no information was lost because information based on the expression of the excluded gene sequences is still represented by sequences in the subset. Therefore, expression of the gene sequences of the subset has information content relevant to properties and/or characteristics of cells generally, including those beyond the plurality of known classes used to generate the original gene expression data set.

Without being bound by theory, and offered to improve the understanding of the invention, some of the redundancy in gene expression information is believed to be due to many gene sequences being co-expressed because they participate in the same biochemical or regulatory pathways within a cell. The invention, by removing or reducing the inclusion of redundant gene sequences from expression profiling, provides the advantage of decreasing, reducing, minimizing, or avoiding the contribution of “noise” (or error) from redundant sequences that are ultimately uncorrelated with the classes to be classified by the subset. Additionally, the invention provides the advantage of reducing over representation, due to certain pathways having significantly higher numbers of redundant gene sequences that are co-expressed than other pathways with relatively few (or no) redundant gene sequences that are co-expressed. Decreasing overrepresentation is important to any classification algorithm. As a non-limiting example where a supervised classification algorithm like KNN is used, the classification is conducted in part based upon the identification of the “k nearest neighbors”. So where the “nearest neighbors” are identified based upon redundant expression information of many or all gene sequences in one pathway, the accuracy of the classification can be significantly reduced. As an additional non-limiting example, if a clustering algorithm is used, then over represented gene sequences would tend to “crowd out” other gene sequences during clustering.

Moreover, and based upon the above non-limiting interpretation, it is believed that the expression data for a range of about 200 to about 6000 gene sequences are sufficient to represent the majority, if not all, possible cellular phenotypes because the remaining gene sequences of a cell's transcriptome provide redundant gene expression information. The range of about 200 to about 6000 gene sequences is based on the following Table 1.

TABLE 1

| | |
|---|---|
| Accuracy | Number of |

| r | Cross-validation | Prediction | Genes |
|---|---|---|---|
| 1.00 | 0.791 | 0.777 | 16948 |
| 0.95 | 0.791 | 0.787 | 16748 |
| 0.90 | 0.794 | 0.787 | 16480 |
| 0.85 | 0.794 | 0.787 | 16069 |
| 0.80 | 0.797 | 0.798 | 15436 |
| 0.75 | 0.805 | 0.798 | 14597 |
| 0.70 | 0.805 | 0.787 | 13477 |
| 0.65 | 0.805 | 0.787 | 12039 |
| 0.60 | 0.826 | 0.777 | 10295 |
| 0.55 | 0.826 | 0.777 | 8220 |
| 0.50 | 0.845 | 0.798 | 6118 |
| 0.45 | 0.850 | 0.809 | 4113 |
| 0.40 | 0.845 | 0.840 | 1935 |
| 0.35 | 0.850 | 0.819 | 1211 |
| 0.30 | 0.848 | 0.819 | 517 |
| 0.25 | 0.802 | 0.798 | 213 |
| 0.20 | 0.719 | 0.670 | 91 |

Table 1 shows the results of an embodiment of the invention wherein the gene expression data of 16,948 distinct gene sequences were subjected to the above methods to reduce redundant gene sequences. The expression of each gene sequence was correlated with each other sequence, and the variance in expression across all samples (374 samples of 34 different tumor types) was determined. Accuracy under LOOCV conditions and prediction conditions of 94 other samples as a testing set with various values of r (Pearson's correlation coefficient) are shown. Where r=1, only each gene sequence within the 16,948 set satisfy the criterion and so the expression of all gene sequences was used to determine accuracy.

As evident from Table 1, accuracy unexpectedly increases with the use of few sequences until an apparent peak at r=0.40 or thereabouts. That corresponds to about 1935 gene sequences. Interestingly, the performance of gene sets ranging from about 200 to about 6000 gene sequences was about the same or better than much larger gene sets, such as the entire set of 16,948 sequences.

As noted above, the plurality of known classes used in the above methods to reduce redundant gene sequences is preferably at least 10, at least 20, at least 30, or at least 40 or more different types of cells with different properties and/or characteristics, including normal and/or diseased related cells. The plurality may include those of different tissue types as well as subtypes of cancer and classes of other diseases, such as among different diseases of similar phenotypes (such as, but not limited to, senile dementia and Alzheimer's disease) or among different types or subtypes of the same disease (such as, but not limited to, psoriasis).

In other embodiments, gene expression data from samples of non-tumor (or normal) cells of known classes are included with tumor samples in a gene expression data set such that the resultant subset may also classify a test sample as a non-tumor (normal) class or a tumor class as the case may be. The non-tumor cells of known classes may be of the same origin as the tumor samples of known classes. It is not necessary, however, for normal cells to be included to provide the ability to classify normal cells. The invention also provides methods for identifying a subset of gene sequences expressed in a cell to classify a cell containing sample as tumor or non-tumor (normal); and if classified as tumor, as being from a particular cell or tissue and/or as a subtype of tumor from the same origin. While normal cells may also be used in embodiments of the invention for generation of a subset of gene sequences, the inclusion of normal cells is not necessarily critical to classification among normal cells of different classes (like tissue type) because a plurality of non-normal cell samples can permit the identification of expressed gene subsets with sufficient gene expression information relating to a phenotype like tissue type.

The gene expression data used to obtain a subset of gene sequences expressed in a cell may be of any suitable form. In some embodiments of the invention, the data is that from a microarray or bead based array used to analyze gene expression in a cell containing sample of any type described herein. Non-limiting examples include gene expression data collected from frozen samples and formalin fixed and paraffin embedded (FFPE) samples. The data may be generated by detection of expressed sequences (such as mRNA) in cells of the samples, optionally with or without amplification of the expressed sequences, followed by their detection by hybridization to a microarray or a bead based array under suitable conditions. The probes used on the microarray to detect the expressed sequences may be cDNA probes, oligonucleotide probes, or any other suitable probe for detecting the expressed sequences as described herein. The levels of gene expression detected by use of a microarray or bead array is the data used in some embodiments of the invention. In other embodiments, the gene expression data may be obtained in the form of proteomic information based on the expression (or presence) of certain polypeptides or fragments thereof. In yet additional embodiments, the data may be from the results of comparative genomic hybridization (CGH). Gene expression data from a combination of sources may of course also be used. The gene expression data may also be optionally processed, such as by filters or comparisons to average signals or expression of a “control” or “housekeeping gene” or another gene analyzed by the microarray before use in the present invention.

The number of classes of known tumor samples depends upon the purposes and goals of the skilled person. But in many embodiments of the invention, the number of classes is based upon the number of available known samples of each class available for training. Thus in some embodiments of the invention, only classes with 3 or more, 4 or more, 5 or more, 6 or more, 7 or more, 8 or more, 9 or more, or 10 or more samples per class are used. Of course it is not necessary that every class be based upon the same number of samples. The known samples may be those which have been classified by standard pathology methods, such as based upon histology and/or cytology. Thus the number of classes is only limited by the availability of samples. The invention may thus be utilized to identify subsets of expressed gene sequences for 3 or more, 5 or more, 7 or more, 9 or more, 10 or more, 12 or more, 14 or more, 16 or more, 18 or more, 20 or more, 25 or more, 30 or more, 35 or more, 40 or more, 45 or more, 50 or more, 55 or more, or 60 or more classes given the availability of known samples of each class.

In another aspect of the invention, a subset of gene sequences, such as those obtained by practice of the above described method is provided. The subset of sequences are those expressed in a cell, wherein the subset has reduced redundancy and the expression of said sequences is greater in variability than that of a larger set of sequences containing said subset. In some embodiments, the subset comprises about 200 to about 6000 gene sequences, the expression of which can classify cells among a plurality of classes, optionally including a property and/or characteristic beyond those of the cells used to obtain the subset. In other embodiments, a subset comprises about 300, about 400, about 500, about 600, about 700, about 800, about 900, about 1000, about 1500, about 2000, about 2500, about 3000, about 3500, about 4000, about 4500, about 5000, or about 5500 or more gene sequences expressed in a cell. In further embodiments, a subset comprises about 200 to about 5000, about 500 to about 4000, or about 1000 to about 3000 gene sequences. Optionally subsets of about 4000 or less (but more than about 200) gene sequences are used. A subset of gene sequences may also be considered a gene set, comprising a fraction, or subset, of all transcripts expressed by a cell, that provides information useful for classification.

A subset of gene sequences provided by the practice of the invention is one wherein i) expression of each gene in the set has a correlation coefficient r ranging from about 0.25 to about 0.50 (or an equivalent correlation coefficient thereof) or less with any other gene in the set; ii) the overall variability in expression of gene sequences in the subset in cells of a plurality of classes is higher than the variability in expression of a larger second set of gene sequences containing said subset; and iii) gene sequences of said subset may be used to classify cells among a plurality of classes with equal or greater accuracy than a larger second set of gene sequences containing said subset. In some embodiments of the invention, the variability in expression is the variance in expression, such that the overall distribution of variance in expression is shifted higher than that of a larger set of genes comprising the subset (see FIG. 2 for a non-limiting example).

In some embodiments of the invention, the expression of each gene sequence in the set has a correlation coefficient r of about 0.30, about 0.35, about 0.40, about 0.45, about 0.50, or about 0.55 or less with any other gene in the set. In other embodiments, the set of sequences contains from about 1% to about 20%, such as about 2%, about 3%, about 4%, about 5%, about 6%, about 7%, about 8%, about 9%, about 10%, about 11%, about 12%, about 13%, about 14%, about 15%, about 16%, about 17%, about 18%, or about 19% of the gene sequences expressed in a cell. Embodiments of the invention include those where the gene sets are capable of classifying with about 80% accuracy or more, about 81% accuracy or more, about 82% accuracy or more, about 83% accuracy or more, about 84% accuracy or more, or about 85% accuracy or more.

Given the methodology used to identify a subset of the invention, any given subset has functionally equivalent sets that provided by the invention. These additional sets may simply be ones where one or more gene sequences of a subset is substituted by one or more other sequence with similar correlation and/or variability characteristics. As a non-limiting example, a gene sequence X of a subset may be substituted with another gene sequence, the expression of which is correlated with X and was excluded from the subset because its variance is (slightly) lower than that of X. Such a substituted subset may be considered a revised or altered subset, but it is nevertheless able to function as a subset of the invention. While the degree or amount of substitution may be limited by the degradation in classification performance expected with large numbers of replacements with gene sequences with increasing lower variability, a wide range of substituted, revised or altered subsets are specifically contemplated as part of the present invention. One limit to the degree or amount of substitution is the degradation of classification to be the same or less than that available from a larger set of gene sequences comprising the substituted subset. In some embodiments, a substituted subset of the invention may be one with at least about 90%, at least about 92%, at least about 94%, at least about 95%, at least about 96%, at least about 98%, or at least about 99% of the classification performance of the parental unsubstituted subset. A substituted subset of the invention is one where expression, in a cell, of the gene sequences that make up the subset provides the ability to classify in a manner analogous to the parental unsubstituted subset.

A subset of gene sequences of the invention may be embodied in a physical entity of the invention, such as a set of polynucleotide probes or primers used to detect expression of the sequences. Alternatively, the subset of sequences may be embodied as data stored in a storage device of the invention. In the case of polynucleotide embodiments, the probes may either oligonucleotide or cDNA in nature and be part of a physical array, such as, but not limited to, a microarray or bead based array. In other embodiments, the probes may be part of a larger number of probes gene sequences on an array which detects less than about 6000 different expressed sequences, such as less than about 5000, less than about 4000, less than about 3000, less than about 2000, or less than about 1000 but more than about 200 sequences. Optionally, the array may be redundant in that more than one probe is used to detect expression of the same expressed sequence or the same probe is used more than once to provide for the detection of expression of the same expressed sequence.

In a further aspect, a subset of gene sequences expressed in a cell, as provided by the invention, may be used in a method of classifying a cell containing test sample. In some embodiments, the expression of gene sequences in a subset is used with a classification algorithm, such as, but not limited to, the K nearest neighbor algorithm. The classification algorithm is trained with known samples, such as tumor samples as a non-limiting example, used to generate expression data for the sequences in the subset. That data is used as a training set to classify the known samples based upon the gene expression data of each gene sequence in the subset. The trained algorithm is then tested with one or more test samples of known class(es) to evaluate the performance of the trained algorithm in classifying the test sample. In some embodiments, the performance, or accuracy, of a subset is evaluated by leave one out cross validation (LOOCV) using k=5 such that the five “nearest neighbors” (known samples) identified by the trained algorithm in response to the “one left out” test sample are retrieved. If all five identify the test sample correctly, the performance would be 100% with respect to the class of that test sample. If all five incorrectly identify the test sample, then the performance of the gene set would be 0% for that class. In other embodiments, k may be 3, 4, 6, 7, 8, 9, 10, or higher. The performance across all samples in the training set may be used to determine the performance of a subset.

The invention also provides for methods of classifying a cell containing test sample among known classes, such as, but not limited to, tumor classes, by use of a trained classifier algorithm as provided above. The test sample in such methods is usually of a wholly or partially unknown class, such as a clinical sample. Such samples may be freshly isolated samples, frozen samples, or FFPE samples. Fresh samples include those that have been undergone none to little or minimal treatment (such as simply storage at a reduced, non-freezing, temperature). The following discussion is based on tumor samples as a non-limiting exemplification.

The samples may be of a primary tumor sample or of a tumor that has resulted from a metastasis of another tumor. In some circumstances, the tumors may not have undergone classification by traditional pathology techniques, such as, but not limited to, immunohistochemistry based assays, may have been initially classified but confirmation is desired, or have been classified as a “tumor of unknown origin” (TUO) or “unknown primary tumor”. The need for confirmation is particularly relevant in light of the estimates of 5 to 10% misclassification using standard techniques. In some embodiments, the sample contains single cells or homogenous cell populations which have been dissected away from, or otherwise isolated or purified from, contaminating cells beyond that possible by a simple biopsy. Alternatively, undissected cells within a “section” of tissue may be used.

In some embodiments, the method comprises comparing expression of a subset of gene sequences, as provided by the present invention, in a cell containing test sample to expression of the same set in a plurality of tumor samples of known classes. The nature of the tumor samples of known classes may be the same or different from that of the test sample. In some embodiments, the two are the same, such as where tumor samples of known classes that are FFPE in nature (the tumor samples are FFPE samples) to derive a subset of gene sequences which is then used in comparison to a test FFPE sample. In other embodiments, the two are different, such as where tumor samples of known classes that are frozen are used to derive a subset of gene sequences which is then used in comparison to a test FFPE sample.

Comparison may of course be made by use of a trained classifier as provided herein. In some embodiments, the trained classifier is a KNN algorithm trained with known samples as described herein. Alternatively, the comparison may be made by use of the same subset of gene sequences with a different set of known tumor samples, optionally using a different classification algorithm. As a non-limiting example, use of a trained KNN algorithm may be by use of k=5 (or another value as described herein) such that the five “nearest neighbors” or known tumor samples with the closest similarity in gene expression of the subset are used to classify the test sample among the known classes. As provided by the methods described herein, the invention is distinguished from the work of others by the ability to classify a clinical sample among at least 34 tumor types with significant accuracy. In one embodiment, the classification is made based upon detection of gene expression of 1935 genes in a tumor sample to classify it as one of 34 or more tumor types as described herein.

The classification of a sample as being one of the possible tumor types described herein to the exclusion of other tumor types is of course made based upon a level of confidence as described below. Where the level of confidence is low, or an increase in the level of confidence is preferred, the classification can simply be made at the level of a particular tissue origin or cell type for the tumor in the sample. Alternatively, and where a tumor sample is not readily classified as a single tumor type, the invention permits the classification of the sample as one of a few possible tumor types described herein. This advantageously provides for the ability to reduce the number of possible tissue types, cell types, and tumor types from which to consider for selection and administration of therapy to the patient from whom the sample was obtained.

Thus, the invention provides a method of classifying a human tumor sample by detecting the expression levels of a subset of transcribed sequences, provided by the methods disclosed herein, in a nucleic acid containing tumor sample obtained from a human subject, and classifying the sample as one of a plurality of tumor classes as used to train a classifier via expression of the subset of expressed gene sequences. If an increase in the confidence of the classification is preferred, the classification can be adjusted to identify the tumor sample as being of a particular origin or cell type as shown in FIG. 1. Thus an increase in confidence can be made in exchange for a decrease in specificity as to tumor type by identification of origin or cell type.

Based on a resultant cancer classification, the invention provides the advantages of a more accurate identification of a cancer and thus the treatment thereof as well as the prognosis, including survival and/or likelihood of cancer recurrence following treatment, of the subject from whom the sample was obtained. The invention may be advantageously applied to samples of secondary or metastasized tumors, but samples of primary tumors for which the tissue source and tumor type is preferably determined by objective criteria may also be used with the invention.

In some embodiments, gene expression in the test sample is detected by PCR or by other form of amplification, such as, but not limited to, RNA amplification. Multiple alternate means for such analysis are also available, including detection of expression within an assay for global, or near global, gene expression in a sample (e.g. as part of a gene expression profiling analysis such as on a microarray) or by specific detection, such as quantitative PCR (Q-PCR), or real time quantitative PCR. Such methods would utilize one or two primers that are complementary to portions of a gene sequence, where the primers are used to prime nucleic acid synthesis. The newly synthesized nucleic acids are optionally labeled and may be detected directly or by hybridization to a polynucleotide of the invention. The newly synthesized nucleic acids may be contacted with polynucleotides (containing gene sequences) of the invention under conditions which allow for their hybridization. Additional methods to detect the expression of expressed nucleic acids include RNAse protection assays, including liquid phase hybridizations, and in situ hybridization of cells.

The expression of the gene sequences of a set identified by the methods of the invention in a test sample may be determined and compared to the expression of said sequences in reference data of non-normal or cancerous cells. Alternatively, the expression level may be compared to expression levels in normal or non-cancerous cells, such as, but not limited to, those from the same sample or subject. In embodiments of the invention utilizing Q-PCR or real time Q-PCR, the expression level may be compared to expression levels of reference genes in the same sample or a ratio of expression levels may be used.

Another embodiment using a nucleic acid based assay to determine expression is by immobilization of one or more gene sequences on a solid support, including, but not limited to, a solid substrate as an array or to beads or bead based technology as known in the art. Alternatively, solution based expression assays known in the art may also be used. The immobilized gene sequences may be in the form of polynucleotides that are unique or otherwise specific to the gene sequences of a subset such that the polynucleotides would be capable of hybridizing to the DNA or RNA of said genes. These polynucleotides may be the full length of the gene sequences or be short sequences of the genes (up to one nucleotide shorter than the full length sequence known in the art by deletion from the 5′ or 3′ end of the sequence) that are optionally minimally interrupted (such as by mismatches or inserted non-complementary basepairs) such that hybridization with a DNA or RNA corresponding to the genes is not affected. As non-limiting examples, the polynucleotides used are from the 3′ end of the gene, such as within about 350, about 300, about 250, about 200, about 150, about 100, or about 50 nucleotides from the polyadenylation signal or polyadenylation site of a gene or expressed sequence.

As will be appreciated by those skilled in the art, some gene sequences include 3′ poly A (or poly T on the complementary strand) stretches that do not contribute to the uniqueness of the disclosed sequences. The invention may thus be practiced with gene sequences lacking the 3′ poly A (or poly T) stretches. The uniqueness of sequences refers to the portions or entireties of the sequences which are found only in nucleic acids, including unique sequences found at the 3′ untranslated portion thereof, of the sequence. Some unique sequences for the practice of the invention are those which contribute to the consensus sequences for the genes such that the unique sequences will be useful in detecting expression in a variety of individuals rather than being specific for a polymorphism present in some individuals. Alternatively, sequences unique to an individual or a subpopulation may be used.

In some embodiments of the invention, polynucleotides having sequences present in the 3′ untranslated and/or non-coding regions of gene sequences of a subset are used to detect expression levels in cancer cells in the practice of the invention. Such polynucleotides may optionally contain sequences found in the 3′ portions of the coding regions of gene sequences. Polynucleotides containing a combination of sequences from the coding and 3′ non-coding regions may have the sequences arranged contiguously, with no intervening heterologous sequence(s).

Alternatively, the invention may be practiced with polynucleotides having sequences present in the 5′ untranslated and/or non-coding regions of gene sequences to detect the level of expression in cancer cells. Such polynucleotides may optionally contain sequences found in the 5′ portions of the coding regions. Polynucleotides containing a combination of sequences from the coding and 5′ non-coding regions may have the sequences arranged contiguously, with no intervening heterologous sequence(s). The invention may also be practiced with sequences present in the coding regions of gene sequences.

In some embodiments, polynucleotides contain sequences from 3′ or 5′ untranslated and/or non-coding regions of at least about 16, at least about 18, at least about 20, at least about 22, at least about 24, at least about 26, at least about 28, at least about 30, at least about 32, at least about 34, at least about 36, at least about 38, at least about 40, at least about 42, at least about 44, or at least about 46 consecutive nucleotides. The term “about” as used in the previous sentence refers to an increase or decrease of 1 from the stated numerical value. Other polynucleotides containing sequences of at least or about 50, at least or about 100, at least about or 150, at least or about 200, at least or about 250, at least or about 300, at least or about 350, or at least or about 400 consecutive nucleotides. The term “about” as used in the preceding sentence refers to an increase or decrease of 10% from the stated numerical value.

Sequences from the 3′ or 5′ end of gene coding regions as found in polynucleotides of the invention are of the same lengths as those described above, except that they would naturally be limited by the length of the coding region. The 3′ end of a coding region may include sequences up to the 3′ half of the coding region. Conversely, the 5′ end of a coding region may include sequences up the 5′ half of the coding region. Of course the entire sequences, or the coding regions and polynucleotides containing portions thereof, may be used in their entireties.

In another embodiment of the invention, polynucleotides containing deletions of nucleotides from the 5′ and/or 3′ end of gene sequences may be used. The deletions may be of 1-5, 5-10, 10-15, 15-20, 20-25, 25-30, 30-35, 35-40, 40-45, 45-50, 50-60, 60-70, 70-80, 80-90, 90-100, 100-125, 125-150, 150-175, or 175-200 nucleotides from the 5′ and/or 3′ end, as non-limiting examples, although the extent of the deletions would naturally be limited by the length of the sequences and the need to be able to use the polynucleotides for the detection of expression levels.

Other polynucleotides of the invention from the 3′ end of gene sequences include those of primers and optional probes for quantitative PCR. In some embodiments, the primers and probes are those which amplify a region less than about 350, less than about 300, less than about 250, less than about 200, less than about 150, less than about 100, or less than about 50 nucleotides from the from the polyadenylation signal or polyadenylation site of a gene or expressed sequence.

Other polynucleotides for use in the practice of the invention include those that have sufficient homology to gene sequences of a subset. Such polynucleotides may have about or 95%, about or 96%, about or 97%, about or 98%, or about or 99% identity with the gene sequences to be used. Identity is determined using the BLAST algorithm, as described above. Other polynucleotides for use in the practice of the invention may also be described on the basis of the ability to hybridize to gene sequences of a subset under stringent conditions of about 30% v/v to about 50% formamide and from about 0.01M to about 0.15M salt for hybridization and from about 0.01M to about 0.15M salt for wash conditions at about 55 to about 65° C. or higher, or conditions equivalent thereto.

In a further embodiment of the invention, a population of single stranded nucleic acid molecules comprising one or both strands of human gene sequence(s) of a subset is provided as a probe such that at least a portion of said population may be hybridized to one or both strands of a nucleic acid molecule quantitatively amplified from RNA of a cancer cell. The population may be only the antisense strand of a human gene sequence such that a sense strand of a molecule from, or amplified from, a cancer cell may be hybridized to a portion of said population. The population may comprise a sufficiently excess amount of said one or both strands of a human gene sequence in comparison to the amount of expressed (or amplified) nucleic acid molecules containing a complementary gene sequence.

The ability to classify is conferred by the identification of expression of the individual gene sequences as relevant and not by the form of the assay used to determine the actual level of expression. An assay may utilize any identifying feature of an identified individual gene as disclosed herein as long as the assay reflects, quantitatively or qualitatively, expression of the gene in the “transcriptome” (the transcribed fraction of genes in a genome) or the “proteome” (the translated fraction of expressed genes in a genome) of a cell. Additional assays include those based on the detection of polypeptide fragments of the relevant member or members of the proteome. Identifying features include, but are not limited to, unique nucleic acid sequences used to encode (DNA), or express (RNA), said gene or epitopes specific to, or activities of, a protein encoded by said gene. Thus the invention may be practiced by detection of gene expression, whether embodied in nucleic acid expression, protein expression, or other expression formats.

So an assay of the invention may utilize a means related to the expression level of the sequences disclosed herein as long as the assay reflects, quantitatively or qualitatively, expression of the sequence. In some embodiments, a quantitative assay means is used. The ability to determine cancer type is provided by the recognition of the relevancy of the level of expression of the sequences in the subset and not by the form of the assay used to determine the actual level of expression. Identifying features of the sequences include, but are not limited to, unique nucleic acid sequences used to encode (DNA), or express (RNA), the disclosed sequences or epitopes specific to, or activities of, proteins encoded by the sequences. Alternative means include detection of nucleic acid amplification as indicative of increased expression levels and nucleic acid inactivation, deletion, or methylation, as indicative of decreased expression levels. Stated differently, the invention may be practiced by assaying one or more aspect of the DNA template(s) underlying the expression of the disclosed sequence(s), of the RNA used as an intermediate to express the sequence(s), or of the proteinaceous product expressed by the sequence(s), as well as proteolytic fragments of such products. As such, the detection of the presence of, amount of, stability of, or degradation (including rate) of, such DNA, RNA and proteinaceous molecules may be used in the practice of the invention.

In some embodiments of the invention, gene expression may be determined by analysis of expressed protein in a cell sample of interest by use of one or more antibodies specific for one or more epitopes of individual gene products (proteins), or proteolytic fragments thereof, in said cell sample or in a bodily fluid of a subject. Such antibodies may be labeled to permit their easy detection after binding to the gene product. Detection methodologies suitable for use in the practice of the invention include, but are not limited to, immunohistochemistry of cell containing samples or tissue, enzyme linked immunosorbent assays (ELISAs) including antibody sandwich assays of cell containing tissues or blood samples, mass spectroscopy, and immuno-PCR.

The practice of the present invention is unaffected by the presence of minor mismatches between the sequences used to identify a gene sequence of a subset and those expressed by cells of a subject's sample. A non-limiting example of the existence of such mismatches are seen in cases of sequence polymorphisms between individuals of a species, such as individual human patients within Homo sapiens. Knowledge that expression of the sequences of a subset (and sequences that vary due to minor mismatches) is correlated with a particular cancer type is sufficient for the practice of the invention with an appropriate cell containing sample via an assay for expression.

The classification of a clinical sample may also be practiced by analyzing gene expression from single cells or homogenous cell populations which have been dissected (or microdissected) away from, or otherwise isolated or purified from, contaminating cells of a sample as present in a simple biopsy. Non-limiting samples of the invention are isolated via non-invasive or minimally invasive means. The expression of genes in said unknown sample is determined and compared to the expression of the subset of genes in reference data used to train a classifier as disclosed herein. The practice of the invention to classify an unknown tumor sample as a tumor type may be by use of an appropriate classification algorithm that utilizes supervised learning to accept 1) the levels of expression of the sequences in a subset in a plurality of known tumor types as a training set and 2) the levels of expression of the same genes in one or more cells of a tumor sample to classify the sample as one of the tumor types.

One advantage provided by the present invention is that contaminating, non-tumor cells (such as infiltrating lymphocytes or other immune system cells) may be removed as so be absent from affecting the genes identified or the subsequent analysis of gene expression to classify cancer. Such contamination is present where a biopsy is used to obtain a subset of the invention.

The detection of gene expression may be conducted in a test FFPE sample, such as that of an unknown tumor. As described herein, the detection may comprise detecting the expression of all or part of the transcribed sequences of a subset, such as by amplification of all or part of the transcribed sequences. In some embodiments of the invention, the amplification comprises linear RNA amplification or quantitative PCR, such as of sequences present within 300 nucleotides of the polyadenylation sites of the transcripts. The use of quantitative PCR amplification of at least 50 nucleotides of the transcripts is one embodiment of the invention. Such amplification may be done at least in part via a multiplex QPCR protocol. Application of such a protocol, or combinations of protocols, to detect expression of a predictor set of genes as provided by the methods of the invention is a multiplex QPCR detection system of the invention. Alternatively, the detection may be based on a microarray comprising oligonucleotide probes to detect the expression of the genes, such as by detection of amplification products thereof.

Alternatively, the expression of gene sequences in FFPE samples may be detected as disclosed in U.S. applications 60/504,087, filed Sep. 19, 2003, 10/727,100, filed Dec. 2, 2003, and 10/773,761, filed Feb. 6, 2004 (all three of which are hereby incorporated by reference as if fully set forth). Briefly, the expression of all or part of an expressed gene sequence or transcript may be detected by use of hybridization mediated detection (such as, but not limited to, microarray, bead, or particle based technology) or quantitative PCR mediated detection (such as, but not limited to, real time PCR and reverse transcriptase PCR) as non-limiting examples. The expression of all or part of an expressed polypeptide may be detected by use of immunohistochemistry techniques or other antibody mediated detection (such as, but not limited to, use of labeled antibodies that bind specifically to at least part of the polypeptide relative to other polypeptides) as non-limiting examples. Additional means for analysis of gene expression are available, including detection of expression within an assay for global, or near global, gene expression in a sample (e.g. as part of a gene expression profiling analysis such as on a microarray). Non-limiting examples those described in U.S. patent application Ser. No. 10/062,857 (filed on Oct. 25, 2001), as well as U.S. Provisional Patent Applications 60/298,847 (filed Jun. 15, 2001) and 60/257,801 (filed Dec. 22, 2000), all of which are hereby incorporated by reference in their entireties as if fully set forth.

The invention also provides computer implemented embodiments of the methods described herein. In some embodiments, the computer shown in FIG. 5 may be configured for use in the practice of the invention. With respect to FIG. 5, the various elements within the computer 200 can be coupled using one or more computer busses 202. The one or more storage devices 268 can include, but are not limited to, ROM, RAM, non-volatile RAM, flash memory, magnetic storage, optical storage, tape storage, hard disk storage, and the like, or some other form of processor readable medium. The memory 224 and the storage devices 268 can include one or more processor readable instructions stored as software. The software can be configured to direct the processor 220 to perform some or all of the functions within the computer 110 within the system 100 of FIG. 5. The software can include stand alone software executed by the processor 220, or the software can run within an operating system or within another software program.

Of course, not every computer 200 includes all of the modules or elements depicted in the embodiment of FIG. 5. Some of the elements are optional and may be omitted. Other elements not shown can be added to the computer 200.

Having now generally described the invention, the same will be more readily understood through reference to the following examples which are provided by way of illustration, and are not intended to be limiting of the present invention, unless specified.

### EXAMPLES

### Example 1

### Materials and Methods

The following Table 2 shows the types and number of samples of known tumors used in the examples that follow. Generally, the 500 samples were fresh or frozen samples of tumor containing tissue. The 468 samples (covering 38 tumor types) were used for further experiments by talking 374 as the training set and the remaining 94 samples as the testing set. Tumor types of fewer than 5 samples were not used initially.

TABLE 2

| Tumor type | Number of samples |
|---|---|
| Adrenal | 7 |
| Brain-glial | 16 |
| Brain-Meningioma | 7 |
| Breast | 43 |
| Cervix-adeno | 8 |
| Cervix-squamous | 13 |
| Endometrium | 13 |
| GallBladder | 5 |
| Germ-cell | 22 |
| GIST | 10 |
| Kidney | 11 |
| Leiomyosarcoma | 13 |
| Liver | 14 |
| Lung-adeno | 9 |
| Lung-large | 9 |
| Lung-small | 8 |
| Lung-squamous | 10 |
| Lymphoma-B | 7 |
| Lymphoma-Hodgkins | 9 |
| Lymphoma-T | 5 |
| Mesothelioma | 10 |
| Osteosarcoma | 7 |
| Ovary-clear | 14 |
| Ovary-serous | 14 |
| Pancreas | 24 |
| Prostate | 11 |
| Skin-basal-cell | 5 |
| Skin-melanoma | 10 |
| Skin-squamous | 6 |
| Small-and-large-bowel | 42 |
| Soft-tissue-Liposarcoma | 5 |
| Soft-tissue-MFH | 11 |
| Soft-tissue-Sarcoma-synovial | 7 |
| Stomach-adeno | 9 |
| Testis-Seminoma | 10 |
| Thyroid-follicular-papillary | 12 |
| Thyroid-medullary | 7 |
| Urinary Bladder | 25 |
| Total | 468 |
| Bile-Duct | 1 |
| Cholangiocarcinoma | 4 |
| Esophagus | 2 |
| Esophagus-Barretts | 4 |
| Esophagus-squamous | 4 |
| HN-squamous | 3 |
| Ovary (unclassified) | 1 |
| Ovary-endometriod | 1 |
| Ovary-mucinous | 4 |
| Ovary-stromal | 1 |
| Soft-tissue-Ewings-sarcoma | 2 |
| Soft-tissue-Fibrosarcoma | 2 |
| Soft-tissue-Rhabdomyosarcoma | 3 |
| Total | 32 |

The samples contained both primary and metastatic tumors with a confirmed diagnosis. A single 5 μm section was stained (H+E), and the tumor visualized. Pure tumor populations were obtained by either manual dissection, or laser capture microdissection (Arcturus, Mountain View, Calif.).

RNA extraction and quality control were performed on each sample. Briefly, samples were processed using a silica spin column-based extraction method (Arcturus, Mountain View, Calif.). The total quantity of RNA extracted was assessed using quantitative PCR (Taqman, ABI), with primers specific for β-actin transcription. Only samples with greater than 10 ng of RNA were amplified.

Samples were amplified using a modified RNA polymerase 2-round amplification protocol (Arcturus, Mountain View, Calif.). Following amplification, the RNA product yield was quantitated by OD (260/280) spectroscopy, and the amplified product visualized by agarose (2%) denaturing gel electrophoresis.

The amplified product from each sample was then hybridized to a microarray containing probes to provide expression data with respect to 16,948 gene sequences. Random gene selection was performed using random sampling function software. For each number of genes selected, random samples were selected 100 times and used to compute the cross-validation and predictive accuracies on both training and testing sets. Corrtrim was performed as described herein. Cross-validation was by dividing the training set into parts with one being used to train and another being used as a test.

### Example 2

### Initial Observations

The mean of the accuracies from 100 random samplings (each step from 50 to 16,948 genes) as well as the gene sets shown in Table 1 (Corrtrim), and the 95% confidence interval for each, were calculated and plotted as shown in FIG. 3. The plots show the cross-validation and predictive accuracies from use of the KNN (k-nearest neighbor) algorithm versus the number of gene sequences used for training and classification.

As evident from the Figure, sets of gene sequences obtained by the method of the present invention had improved accuracy in comparison to randomly sequences selected sequences. Moreover, the sets with about 200 to about 6000 gene sequences had accuracies equal to or greater than using the totality of nearly 17,000 genes. Similar results are observed with the use of known FFPE tumor specimens samples and KNN after extraction of RNA which was analyzed for gene expression.

### Example 3

### Confirmation of Observation

To confirm that the results seen in FIG. 3 are not the result of an effect at an arbitrary threshold present in the method used, successive removal of gene sequences was conducted as follows. At each step of the Corrtrim method, the best correlation coefficient r, determined based upon cross-validation accuracies using the KNN method is determined. the expression data for the k selected gene sequences were then removed from the data set, and the remaining data used to enter the next round of gene selection. Successive rounds of gene selection stopped when the remaining number of gene sequences was less than 100. The results for the first four rounds of successive selection are shown in FIG. 4.

As seen in FIG. 4, performance of the gene sequences at best correlation coefficient value progressively drops after each round, indicating that Corrtrim does not produce one of a number of different sets of gene sequences with identical performance capabilities in classification.

### Example 4

### Comparison of the Invention to Other Methods

A comparison based on the classification of three different cell types demonstrates the efficiency of the methods of the invention in comparison to methods using either supervised gene selection or clustering. If classification among classes A, B, and C is desired, a method to reduce the number of genes used to classify among the classes may first identify gene sequences expressed in correlation with each class. This may be done by supervised training or by clustering. If gene groups 1, 2, and 3 are identified as expressed in correlation with each of classes A, B, and C, respectively, the next step would be the removal of redundant gene sequences, such as by a simple t test or analogous method. Thus genes X, Y, and Z (from groups A, B, and C, respectively) may be identified as being the sequences expressed best for the classification of A, B, and C, respectively.

In contrast, the methods of the invention do not include an initial correlation of gene expression with each of A, B, and C. Thus if the same classes and expression data are used and genes X and Y are correlated to each other at r=0.55, and Z is independently correlated with each of X and Y at r=0.2, use of the present invention and a correlation coefficient of r=0.4 would result in a subset with only one of X and Y (but not both because only the one with the greater variance overall would be in the subset) while gene Z would be in the subset. Thus the methods of the invention would result in a subset of gene sequences, such as the 1935 set in Table 1, which would include genes X and Z which still being able to classify among classes A, B, and C, by use of genes X and Z (and perhaps other sequences in the subset). Thus, the instant methods result in a more efficient set of gene sequences for examination to classify A, B, and C. Additionally, the methods of the invention would not result in the same gene set as methods using either supervised gene selection or clustering as shown above.

### Example 5

### Ability to Classify Additional Classes

As described herein, the invention may be used to provide a subset of gene sequences, the expression of which may be used to classify cell types (or phenotypes) used to derive the subset as well as to classify additional cell types (or phenotypes) not used in obtaining the subset. This is readily demonstrated using a variation of Example 1 herein as follows.

The samples of Example 1, covering 34 different sample types of more than 5 samples each, are divided into a first group of 30 sample types and a second group of 4 sample types. Expression data from the first group of 30 sample types are treated by the methods as described herein to arrive at a subset of gene sequences, from the 16,948 set, which best classifies the 30 sample types based upon LOOCV using KNN. The expression of the subset of gene sequences is used to classify the samples of the combined first and second groups using KNN and LOOCV among the combined samples. Significant accuracy in classifying the samples of the second group is seen.

### Example 6

### Classification of Tumor Origin

The classification of a tumor sample as one of the 38 tumor types of Example 1 inherently also classifies the tissue or organ site origin of the sample. For example, the identification of a sample as being cervix-squamous necessarily classifies the tumor as being of cervical origin, squamous cell type (and thus epithelial rather than non-epithelial in origin) as shown in FIG. 1. It also means that the tumor was necessarily not germ cell in origin. Thus, the methods of the invention may be applied to classification of a tumor sample as being of a particular tissue or organ site of a subject or patient. This application of the invention is particularly useful in cases where the sample is of a tumor that is the result of metastasis by another tumor.

The KNN algorithm may be used to perform the above classification. KNN can be used to analyze the expression data of a subset of gene sequences in a “training set” of known tumor samples including all 38 of the tumor types in Example 1. The training data set can then be compared to the expression data for the same genes in an unknown tumor sample. The expression levels of the genes in the unknown tumor sample are then compared to the training data set via KNN to identify those tumor samples with the most similar expression patterns. As a non-limiting example, the five “nearest neighbors” may be identified and the tumor types thereof used to classify the unknown tumor sample. Of course other numbers of “nearest neighbors” may be used.

As a hypothetical example, if the five “nearest neighbors” of an unknown sample are four B cell lymphomas and one T cell lymphoma, then the classification of the sample as being of a B cell lymphoma can be made with great accuracy.

The classification ability may be combined with the inherent nature of a classification scheme (like that of FIG. 1) to provides a means to increase the confidence of tumor classification in certain situations. For example, if the five “nearest neighbors” of an unknown sample are three ovary clear cell and two ovary serous tumors, confidence can be improved by simply treating the tumors as being of ovarian origin and treating the subject or patient (from whom the sample was obtained) accordingly. See FIG. 1. This is an example of trading off specificity in favor of increased confidence. This provides the added benefit of addressing the possibility that the unknown sample was a mucinous or endometroid tumor. Of course the skilled practitioner is free to treat the tumor as one or both of these two most likely possibilities and acting accordingly.

Because the developmental lineage of tumor cells in certain tumor types (e.g., germ cells) can be complex and involve multiple cell types, FIG. 1 may appear to be oversimplified. However, it serves as a good basis to relate known histopathology and to serve as a “guide tree” for analyzing and relating tumor-associated gene expression signatures.

The inherent nature of the classification scheme also provides a means to increase the confidence of tumor classification in cases wherein the “nearest neighbors” are ambiguous. For example, if the five “nearest neighbors” were one urinary bladder, one breast, one kidney, one liver, and one prostate, the classification can simply be that of a non-squamous cell tumor. Such a determination can be made with significant confidence and the subject or patient from whom the sample was obtained can be treated accordingly. Without being bound by theory, and offered solely to improve the understanding of the invention, the last two examples are believed to reflect the similarities in gene expression of cells of a similar cell type and/or tissue origin.

All references cited herein, including patents, patent applications, and publications, are hereby incorporated by reference in their entireties, whether previously specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

1. A method of reducing the number of gene sequences required for use in classifying cells or cell containing tissues based upon the expression of said sequences, said method comprising
providing a gene expression data set of gene sequences expressed in a plurality of cell containing samples of a plurality of classes,
determining the range of variability in expression of each gene sequence across the plurality of samples;
correlating, across the plurality of samples, the expression level of each gene in the data set with the expression level of each other gene in the data set to produce a correlation matrix of correlation coefficients; and
selecting those gene sequences that are
i) expressed in correlation, below a desired correlation coefficient, with an other gene sequence, and
ii) expressed with greater variability than said other gene sequence,
to result in a subset of genes expressed with higher variability than other gene sequences expressed in correlation with said subset of genes;
wherein the expression of said subset of genes can classify cells among a multitude of classes comprising said plurality of classes.

2. The method of claim 1 wherein said gene sequences expressed in a plurality of cell containing samples provides a gene expression data set of 50% or more of the genes expressed in the transcriptomes of the cells of said plurality of cell containing samples.

3. The method of claim 1 wherein said data set is obtained from array or microarray based analysis of gene expression in said plurality of samples.

4. The method of claim 1 wherein said plurality of classes is 10 or more, and said plurality of samples is 10 or more for each class.

5. The method of claim 1 wherein said plurality of classes includes classes of tumor cells from different tissue types.

6. The method of claim 1 wherein said plurality of classes includes classes of normal cells from different tissue types.

7. The method of claim 1 wherein said desired correlation coefficient is a Pearson's coefficient of about 0.25 or higher.

8. The method of claim 1 wherein said subset of gene sequences is about 200 to about 6000 or more in number.

9. A method of classifying a cell containing test sample among a plurality of known classes, said method comprising comparing expression of the subset of gene sequences identified by the method of claim 1 in a cell containing test sample to expression of said gene sequences in a plurality of samples of known classes; and classifying the test sample as being of one of said known classes.

10. The method of claim 9 wherein said comparison is by use of the KNN algorithm.

11. The method of claim 9 wherein said test sample is a clinical sample, a frozen sample, and an FFPE sample.

12. The method of claim 9 wherein said expression is detected by RT-PCR or by RNA amplification.

13. The method of claim 12 wherein cells of said test sample are isolated by microdissection prior to detection of gene expression.

14. A reduced subset of gene sequences identified or selected by claim 1.

15. An array comprising polynucleotide probes which detect expression of a set of gene sequences comprising about 200 to about 6000 gene sequences, the expression of which can classify cells among a plurality of classes, wherein
i) expression of each gene in the set has a correlation coefficient r ranging from about 0.25 to about 0.50 or less with any other gene in the set,
ii) the overall variability in expression of said set of gene sequences in cells of said plurality of classes is higher than the variability in expression of a larger second set of gene sequences containing said about 200 to about 6000 gene sequences; and
iii) gene sequences of said subset may be used to classify cells among a plurality of classes with equal or greater accuracy than a larger second set of gene sequences containing said about 200 to about 6000 gene sequences.

16. The array of claim 15 which contains from about 1% to about 20% of the gene sequences expressed in a cell.
